(19) United States
(12) Patent Application Publication
(10) Pub. No.: US 2022/0167879 A1
(43) Pub. Date: Jun. 2, 2022

(54) UPPER LIMB FUNCTION ASSESSMENT DEVICE AND USE METHOD THEREOF AND UPPER LIMB REHABILITATION TRAINING SYSTEM AND USE METHOD THEREOF

(71) Applicant: Shenzhen Wisemen Medical Technologies Co., Ltd., Shenzhen City, Guangdong Province (CN)

(72) Inventors: Jianhuang WU, Shenzhen City, Guangdong Province (CN); Wei SUN, Shenzhen City, Guangdong Province (CN)

(21) Appl. No.: 17/432,952

(22) PCT Filed: Oct. 10, 2020
(86) PCT No.: PCT/CN2020/120143

§ 371 Date: Aug. 23, 2021

(30) Foreign Application Priority Data
Jun. 1, 2020 (CN) 202010483531.6

(51) Int. Cl. A61B 5/11; A61H 1/02; A61B 5/00; G16H 20/30

(52) U.S. Cl. CPC A61B 5/1124; A61H 1/0277; A61H 1/0281; A61B 5/1128; A61B 5/4836; A61B 2505/09; A61B 5/0077; A61B 5/742; G16H 20/30; A61H 2201/1659; A61H 2201/1638; A61B 5/7267

(57) ABSTRACT

The present invention provides an upper limb function assessment device and the use method thereof and an upper limb rehabilitation training system and the use method thereof, wherein the upper limb function assessment device includes a display, a depth camera and a central processor, the depth camera is used to capture a user's motion, the display is used to display motion demonstration and the user's motion, and the central processor is connected to the display and the depth camera, respectively. The present invention captures the user's motion precisely with the depth camera, which makes the obtained data more accurate and objective, and also facilitates recording and storage of the obtained data. The central processor determines whether the completion of the motion meets the requirements in assessment scales, allowing the user to come up with an assessment report on his own without requiring a lot of assistance from a physician.

### TECHNICAL FIELD

The present invention relates to the technical field of mechanical rehabilitation physiotherapy equipment, especially to an upper limb function assessment device and its use method thereof and an upper limb rehabilitation training system and its use method thereof.

### BACKGROUND

With the serious phenomenon of aging, the number of stroke patients is increasing, and most of these patients have upper limb dysfunction. Therefore, an upper limb rehabilitation training system is needed to assess the degree of upper limb dysfunction and rehabilitate the upper limb function. There are several international standards for assessing upper limb functional disability, but they are all based on one-to-one assessments for a patient by a physician, through oral instructions, motion perception and recording the execution of motion instructions and perception of muscle strength of the patient, and then filling out a form for assessment. Once a basic determination of the degree of upper limb disability has been made, a diagnostic plan is formulated.

It can be seen that the existing technology uses one-to-one targeted assessment by physicians, and the patient's amplitudes of motion are different at different periods, so it is difficult to judge and record by visual inspection, which will incur certain errors and is not objective. Moreover, the assessment time is long and takes up a lot of time of the physician, resulting in a limited number of patients to be treated each day.

### SUMMARY

The purpose of the present invention is to provide an upper limb function assessment device and its use method thereof and an upper limb rehabilitation training system and its use method thereof to solve the technical problems mentioned above in the prior art.

The present invention provides an upper limb function assessment device, comprising: a display, a depth camera and a central processor, wherein the depth camera is configured to capture a user's motion, the display is configured to display motion demonstration and the user's motion and the central processor is connected to the display and the depth camera, respectively.

Further, the depth camera comprises an RGB camera and a depth camera, wherein the RGB camera is configured to obtain two-dimensional coordinates of the user's joints and the depth camera is configured to obtain depth coordinates of the user's joints.

The present invention further provides an upper limb rehabilitation training system comprising the upper limb function assessment device in the present invention.

Further, the upper limb rehabilitation training system comprises an exoskeleton robotic arm and a motion control unit, wherein the motion control unit is connected to the central processor for controlling the motion of the exoskeleton robotic arm.

Further, the user's motion comprise motion postures of the user's healthy side arm and the depth camera is configured to capture the motion postures of the user's healthy side arm in real time, the central processor controls the motion of the exoskeleton robotic arm according to the motion postures of the user's healthy side arm, thereby driving the user's affected side arm on the exoskeleton robotic arm to make corresponding motion.

Further, the motion control unit controls three drive units for achieving abduction/adduction of the arm, lifting/lowering of the arm and flexion of the forearm of the exoskeleton robotic arm, respectively.

Further, the shoulder joint and the elbow joint of the exoskeleton robotic arm are of a surrounding sliding rail structure.

Further, a plurality of motion scenarios and/or interactive scenarios are stored in the central processor, and the display is configured to display the plurality of motion scenarios and/or interactive scenarios, wherein the plurality of motion scenarios are used for imitation or viewing by the user, and the interactive scenarios are used for interaction with the user.

The present invention further provides a use method of the upper limb function assessment device, comprising the steps of displaying motion demonstration on a display; imitating, by a user, according to the motion demonstration on the display; capturing the user's motion and obtaining three-dimensional coordinates of the user's joints via a depth camera and determining the completion of the user's motion based on the motion demonstration and the imitated motion of the user.

Further, the method comprises retrieving, by means of the central processor, a pre-stored program corresponding to completion differences between the motion demonstration and the imitated motion of the user according to the completion of the user's motion and displaying the program on the display.

The present invention further comprises a use method of the upper limb rehabilitation training system, comprising the steps of: capturing motion postures of the user's healthy side arm by means of a depth camera and deriving coordinate data of the healthy side arm, building a user motion model after the coordinate data is filtered and processed, converting the motion coordinates of the user's healthy side arm to motion coordinates of the affected side arm using a mirror coordinate transformation, calculating motion angles of each joint of the exoskeleton robotic arm by using inverse kinematics solutions and driving, by means of the exoskeleton robotic arm, the user's affected arm to make symmetrical motion with the healthy arm through the execution of the robotic arm servo control system.

Further, the motion postures of the user's healthy arm specifically include one or more of the following: the shoulder joint, the elbow joint and the wrist joint of the healthy arm.

Further, the method comprises detecting the user force and force direction in real time when the exoskeleton robotic arm is in a passive assisted mode and controlling the exoskeleton robotic arm to assist the user in the force direction when the user force exceeds a preset value.

Further, when the exoskeleton robotic arm is in an active training mode, the user's arm drags the robotic arm to drive the exoskeleton robotic arm to move.

The present invention captures the user's motion precisely by means of a depth camera, which makes it possible to obtain more accurate and objective data, and also facilitates recording and storage of the data. By determining whether the completion of the motion meets the requirements in assessment scales, the central processor allows the user to complete the training on his own without requiring a lot of assistance from physicians and can obtain assessment report results of the assessment scales which is commonly used in clinical practice. The assessment time is short and efficient, which greatly reduces the time of physicians.

### BRIEF DESCRIPTION OF THE DRAWINGS

In order to make the technical solution of the invention clear, a brief description of the drawings in the description of the invention will be given below. It is obvious that the drawings in the following description are only some embodiments of the invention, and that other drawings can be obtained from these drawings without creative work for a person skilled in the art.

FIG. 1 is a schematic structural diagram of an upper limb rehabilitation training system provided by an embodiment of the present invention, which shows the upper limb function assessment device of the present invention.

FIG. 2 is a partial enlarged view of FIG. 1, which shows the upper limb rehabilitation training system of the present invention.

FIG. 3 is a schematic diagram of the structure of the exoskeleton robotic arm in FIG. 2;

FIG. 4 is a flowchart of a use method of the upper limb function assessment device according to an embodiment of the present invention;

FIG. 5 is a control principle diagram of the upper limb rehabilitation training system according to an embodiment of the present invention;

FIG. 6 is a flowchart of a use method of the upper limb rehabilitation training system according to an embodiment of the present invention;

FIG. 7 is a control principle diagram of the upper limb rehabilitation training system in a passive rehabilitation training mode according to an embodiment of the present invention.

FIG. 8 is a control principle diagram of the upper limb rehabilitation training system in a passive assisted rehabilitation training mode according to an embodiment of the present invention.

wherein, the above drawings include the following reference signs: 1. medical casters; 2. body; 3. emergency stop switch; 4. push handle; 5. vertical lift module; 6. horizontal motion module; 71. the first joint of the shoulder joint; 72. the second joint of the shoulder joint; 73. the first joint of the elbow joint; 8. the third passive joint of the shoulder joint; 9. the second passive joint of the elbow joint; 10. display; 11. depth camera; 12. display bracket; 13. grip handle; 14. handle puller; 15. arm puller; 16. forearm puller; 17. damped wrist joint; 20. plunger; 101. arm straps; 102. forearm straps

### DETAILED DESCRIPTION

The technical solution of the present invention will be clearly and completely described below in combination of the drawings, and it is clear that the described embodiments are a part of the embodiments of the present invention, and not all of them. Based on the embodiments in the present invention, all other embodiments obtained by a person skilled in the art without creative work fall within the scope of protection of the present invention.

In the description of the present invention, it should be noted that the orientation or position relationship indicated by the terms “center”, “top”, “bottom”, “left” , “right”, “vertical”, “horizontal”, “inside” and “outside” etc. is based on the orientation or position relationship shown in the drawings and is intended only to facilitate and simplify the description of the invention, not to indicate or imply that the device or element referred to must have a particular orientation or be constructed and operate in a particular orientation, and therefore is not to be construed as a limitation of the invention.

In the description of the invention, it is to be noted that, unless otherwise expressly specified and limited, the terms “mounted”, “joined” and “connected” are to be understood in a broad sense. For example, it can be a fixed connection, a detachable connection, or a one-piece connection; it can be a mechanical connection or an electrical connection; it can be a direct connection or an indirect connection through an intermediate medium, and it can also be an interconnection of two components. For a person skilled in the art, the specific meaning of the above terms in the present invention can be understood in the context of specific cases.

In the description of the present invention, an exoskeleton robotic arm is also briefly referred to as a robotic arm, and should be understood as the same meaning for those skilled in the art.

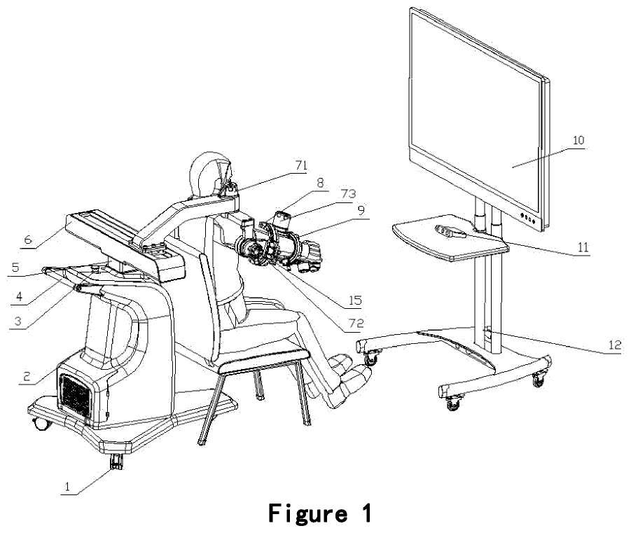

According to one aspect of the present invention, there is provided an upper limb function assessment device, as shown in FIG. 1, the upper limb function assessment device comprises a display 10, a depth camera 11, and a central processor, the depth camera 11 is configured to capture a user's motion, the display 10 is used to display motion demonstration and the user's motion, and the central processor is connected to the display 10 and the depth camera 11, respectively. Preferably, the depth camera 11 is configured to capture information about the angle and position of the user's joints.

As shown in FIG. 1, in this embodiment, the upper limb function assessment device display 10 comprises a display bracket 12, both the display 10 and the depth camera 11 are mounted on the display bracket 12, and rollers are provided at the bottom of the display bracket 12.

The depth camera 11 accurately captures parameters of each joint of the user, and measures information such as the angle and position of the joints to identify and determine a motion range of the user's arm, making data obtained more accurate and objective, and also facilitates the recording and storage of the data. The central processor determines whether the completion of the motion meets the requirements in the assessment scales. The training can be done by the user on his own without requiring a lot of assistance from the physician and can obtain the results of the assessment report of the commonly used assessment scales in clinical practice. The assessment time is short and efficient, which greatly reduces the time of the physicians.

The upper limb function assessment device in the present invention can be used for the assessment and diagnosis of upper limb function disorders caused by stroke and other diseases. On one hand, it solves the physical input of the physicians, on the other hand, it can more objectively and accurately assess the upper limb rehabilitation level of the user having upper limb disability.

In this embodiment, a storage module is provided in the central processor, and a plurality of rehabilitation programs are stored in the storage module, and different assessment results correspond to different rehabilitation programs, and the plurality of assessment results are matched one-to-one with the plurality of rehabilitation programs. The central processor is also used to retrieve the corresponding rehabilitation programs based on the above-mentioned assessment results and display them to the user and/or the physician via the display. This embodiment is also capable of generating targeted assessment reports for reference by the physicians and users, thus enable high assessment efficiency.

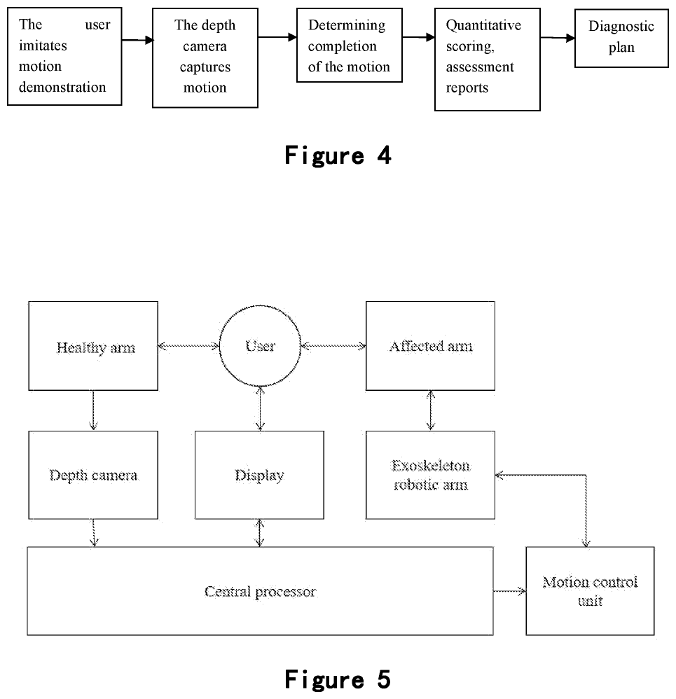

The present invention also provides a method for using the upper limb function assessment device described in the present invention, as shown in FIG. 4, which includes the following steps: displaying a motion demonstration on the display 10, and the user imitates the motion demonstration on the display 10; the depth camera 11 captures the user's motion, and the system obtains three-dimensional coordinates of the user's joints. The central processor determines the completion of the motion (e.g., whether it is fully completed, partially completed, or completely incomplete) based on the motion demonstration and the user's motion to form an assessment report (which can result in the assessment report results of assessment scales which are commonly used in clinical practice), and then the system and the physician provide a diagnosis plan and a targeted exercise prescription. Alternatively, the method includes the following steps: the monitor 10 displays the motion demonstration; the depth camera 11 captures the user's motion, and the system obtains the three-dimensional coordinates of the user's joints; the central processor determines the completion of the user's motion (e.g., whether it is fully completed, partially completed, or completely incomplete) based on the motion demonstration and the user's motion to form an assessment report (which can result in the assessment report results of assessment scales which are commonly used in clinical practice). The system and the physician will then provide a diagnostic plan and a targeted exercise prescription.

The above method further comprises: according to the completion of the user's motion, the central processor retrieves a pre-stored program corresponding to completion differences between the motion demonstration and the imitated motion of the user; and the program is displayed on the display.

Further, the depth camera 11 comprises an RGB camera (red green blue camera) and a depth camera, the RGB camera is used to obtain the 2D coordinates of the user's joints and the depth camera is used to obtain the depth coordinates of the user's joints.

Preferably, the present invention uses the depth camera 11 to capture the 2D postures of the arm without wearing other devices, which is simple and convenient.

Specifically, the user's motion is captured by the depth camera 11, and the system acquires the 3D coordinates of the user's joints. First, a deep learning method based on a deep neural network is adopted to acquire the two-dimensional coordinates of the user's joints from color images captured by the RGB camera of the depth camera 11, and then the depth coordinates of the user's joints are acquired by the depth images captured by the depth camera of the depth camera 11, and finally the acquired two-dimensional coordinates and depth coordinates of the user's joints are mapped to the three-dimensional coordinates of the user's joints. In the deep learning method based on the deep neural network, self-obscuring images and images of special and hard-to-detect motion such as spin-forward and spin-backward are added as training sets to train the deep neural network model. Self-obscuring means that one of the user's joints captured by the depth camera 11 is obscured by other joints of the user itself. For example, when facing the depth camera 11, the three points of the depth camera 11, the wrist joints and the shoulder joints are in a straight line when the forward-extended arm is in a horizontal position, making the shoulder joints at the rear obscured by the wrist joints which leads to inaccurate detection of the joints' coordinates. The advantage of using this method is that it can avoid the problem of inaccurate detection of joints' coordinates due to self-obscuring, and accurately detect the 3D coordinates of the user's joints.

The next step is to determine the completion of the user's motion. The system combines the assessment scales commonly used in clinical practice to perform the assessment by quantifying all the motion in the scales for automatic assessment. The present invention uses a deep learning method based on Long Short-Term Memory to determine the completion of the motion, such as whether it is fully completed, partially completed or completely incomplete. During the training of the model, the key frames and key nodes of different motion sequences are manually marked to achieve static matching of standard motion, followed by automatic sampling to get more adjacent frames to achieve dynamic matching, and the key frames, key nodes and adjacent frames are combined for encoding to form a model of standard motion template sequences. Finally, the longest common subsequence algorithm is used to identify whether the motion performed by the user conforms to the standard motion: firstly, the current motion of the user is detected in real time to form the motion sequence, and then the longest common subsequence is obtained by comparing the motion sequence with the standard motion template sequence model, so as to provide feedback on the non-standard degree of the current motion of the user, and to make judgment and specific scoring for the completion of the motion. The key frames, the key nodes, the adjacent frames and the current motion frames include the 3D coordinates of each joint of the user. After the assessment of all motion is completed, an assessment report is formed, and then the system and the physician provide a diagnosis plan and a targeted exercise prescription.

In addition, during the targeted upper limb rehabilitation training, the treatment plan in the early stage is that the physician moves the user's arm for repetitive rehabilitation training, gradually improving the user's muscle function first, and after rehabilitation to a certain extent, combined with some simple assistive devices for intensive training. Because a large amount of physical input from the physician is required, and the number of patients in China is increasing, the physicians are seriously insufficient, making it difficult for the user to get adequate training.

To this end, the present invention provides an upper limb rehabilitation training system, as shown in FIG. 1 to FIG. 3. The upper limb rehabilitation training system comprises the upper limb function assessment device described above in the present invention, and also comprises an upper limb rehabilitation robot, and the upper limb rehabilitation robot comprises an exoskeleton robotic arm and a motion control unit, and the motion control unit is connected to a central processor for controlling the motion of the exoskeleton robotic arm.

Specifically, the exoskeleton robotic arm comprises a shoulder joint and an elbow joint, the shoulder joint comprises a first joint of the shoulder joint and a second joint of the shoulder joint and a third passive joint of the shoulder joint, and the elbow joint comprises a first joint of the elbow joint and a second passive joint of the elbow joint. The motion control unit controls three drive units for abduction/adduction of the arm, raising/lowering of the arm and flexion of the forearm of the exoskeleton robot arm, respectively. Specifically, the three drive units comprises a first drive unit 71 corresponding to the first joint of the shoulder joint, a second drive unit 72 corresponding to the second joint of the shoulder joint, and a third drive unit 73 corresponding to the elbow joint. The first drive unit 71, the second drive unit 72, and the third drive unit 73 are used to realize the abduction/induction of the arm, the lifting/lowering of the arm, and the flexion of the forearm, respectively. The elbow joint comprises the first joint 73 of the elbow joint and the second passive joint 9 of the elbow joint, and the first joint 73 of the elbow joint is used to realize the flexion of the forearm. Preferably, the third degree of freedom of the shoulder joint of the exoskeleton robotic arm (to achieve flexion of the forearm) is passively controlled.

The upper limb rehabilitation training system provided in this embodiment uses an exoskeleton robotic arm, and when used, the arm is placed inside the exoskeleton robotic arm, which has a large motion range and can realize the motion of the major joints and can effectively perform joint training and rehabilitation. The exoskeleton robotic arm in this embodiment aims at the rehabilitation motion of the three major joints with low cost, simple design and high safety factors. Furthermore, in this embodiment, the robotic arm can be controlled by the motion control unit to drive the upper limb of the user to move, which can solve the problem of muscle strength recovery training for users in the early stage and plays a training role for more serious users in the early stage. It reduces the physical input of physicians and improves the efficiency of rehabilitation training.

As shown in FIG. 3, the central axes of the first drive unit 71, the second drive unit 72 and the third drive unit 73 are orthogonal and two of them are perpendicular to each other. Each drive unit comprises a servo motor, a gearbox, an encoder, a driver and a holding brake, etc., and is an integrated drive unit. Specifically, the drive adopts a hollow integrated drive joint, comprising a servo motor, a harmonic reducer, an incremental encoder, an absolute encoder, a drive controller, a brake and a torque sensor. The drive has the advantage of full function, compact space and convenient wiring.

Preferably, the third passive joint 8 of the shoulder joint and the second passive joint 9 of the elbow joint of the exoskeleton robotic arm adopt a surrounding sliding rail structure. The exoskeleton robotic arm can realize interchange between left and right hands, it can realize fast positioning, switching and fixing in the interchange process of the exoskeleton robotic arm. It only needs to pull out the plunger 20, and automatically positioning and fixing after switching.

As shown in FIG. 3, the exoskeleton robotic arm further comprises an arm puller 15 and a forearm puller 16, and the length of the arm puller 15 and the forearm puller 16 can be adjusted manually or electrically to accommodate users with different arm lengths. The shoulder joint and elbow joint of the user correspond to the position of the shoulder joint and elbow joint of the exoskeleton robotic arm, respectively. In this embodiment, a manual adjustment mechanism is adopted to adjust the spacing among the third passive joint 8 of the shoulder joint, the second passive joint 9 of the elbow joint and the damped wrist joint 17.

Preferably, as shown in FIG. 3, the arm puller 15 is provided with an arm strap 101, and the forearm puller 16 is provided with a forearm strap 102 for binding the user's arm and forearm.

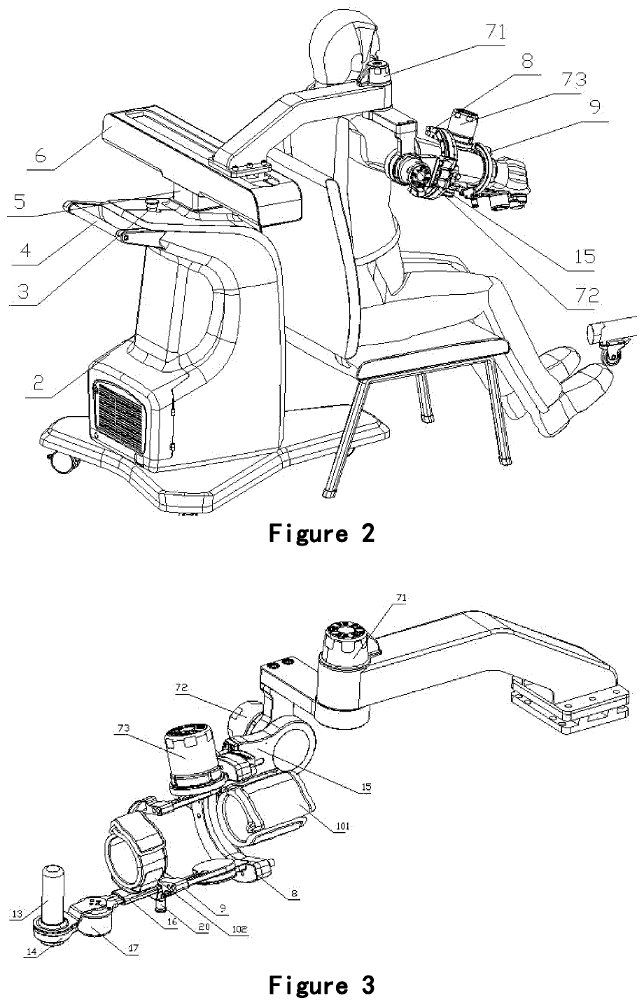

As shown in FIG. 1 and FIG. 2, the upper limb rehabilitation robot in this embodiment further comprises a body 2, a seat on which the user can sit during assessment and training. The exoskeleton robotic arm is mounted on the body 2, which is provided with medical casters 1, an emergency stop switch 3, a push handle 4, a vertical lift module 5, and a horizontal motion module 6. The vertical lift module 5 and the horizontal motion module 6 are respectively used to drive the exoskeleton robotic arm for lifting, translation, and other motion.

As shown in FIG. 3, in this embodiment a damped wrist joint 17 and a grip handle 13 are provided at the end of the exoskeleton robotic arm, which enables measurement and rehabilitation training of the user's wrist and grip strength. The grip handle 13 is connected to the damped wrist joint 17 by a handle puller 14.

The upper limb rehabilitation training system in the present invention is provided with hardware devices such as power supply and a host. In the process of assessment, the host and the display 10 prompt the user to complete various assessment motion through animation, images and sound etc., identify and capture the user's limb motion by means of the depth camera 11, store and record data captured by the depth camera 11, judge the completion of the motion, and provide quantitative scoring results to form an assessment report, which is provided to the physician, who then selects or formulates a diagnosis plan and a targeted exercise prescription.

Preferably, the upper limb rehabilitation training system comprises a solar energy generation device which absorbs solar energy and converts it into electrical energy directly or indirectly through photoelectric effect or photochemical effect for supplying power to the upper limb rehabilitation training system. With the solar energy generation device, the rehabilitation training system of the present invention can be used for rehabilitation training in places where power supply conditions are not available or electrical energy is insufficient, which makes the application range and occasions of the device expanded and gets rid of indoor constraints, and also facilitates the use of new energy sources to achieve energy saving and being eco-friendly.

In one embodiment of the present invention, the solar energy generation device comprises a solar panel, and the solar panel may be provided on the back of the display 10. In another embodiment of the present invention, the solar power generation device comprises a solar thin film cell which is affixed to the outer surface of the upper limb rehabilitation training system.

In particular, when the weather is good, users often go outdoors for rehabilitation training. On one hand, it can effectively relieve the mood and is good for rehabilitation, on the other hand, it can make full use of solar energy which is energy saving and is eco-friendly.

Further, the user's motion comprises motion postures of the user's healthy arm, and the depth camera 11 is used to capture the motion postures of the user's healthy arm in real time; the central processor controls the motion of the exoskeleton robotic arm according to the motion postures of the user's healthy arm, thus driving the affected arm on the exoskeleton robotic arm to make corresponding motion.

Specifically, as shown in FIG. 5, the depth camera, the display and the motion control unit are connected to the central processor, and the motion control unit is connected to the exoskeleton robotic arm. The user's affected arm is located inside the exoskeleton robotic arm when in use. The depth camera is used to capture the motion postures of the user's healthy arm and sends the captured motion posture to the central processor which receives the motion postures of the user's healthy arm and sends instructions to the motion control unit, and the motion control unit controls the exoskeleton robotic arm to perform corresponding motion according to the instructions, the motion control unit controls the motion of the exoskeleton robotic arm according to the instructions, thus driving the motion of the user's affected arm. The display interacts with the central processor bidirectionally and displays the processing information and/or processing results sent by the central processor. Wherein, the motion control unit comprises a robotic arm servo control system, and the robotic arm servo control system is used to control the above motion control unit according to the instructions of the central processor.

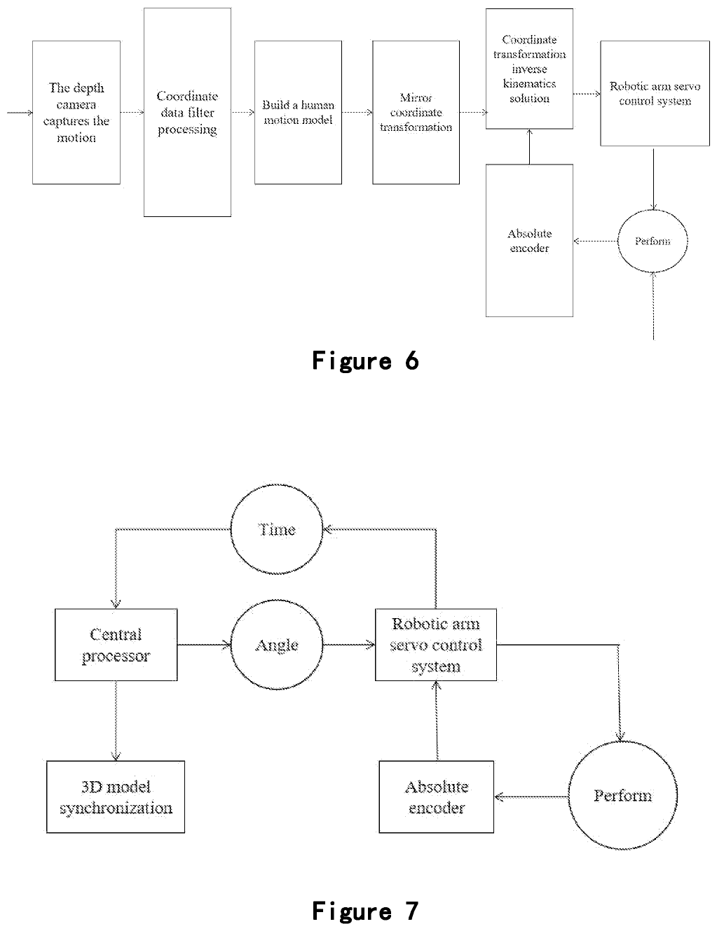

The present invention further provides a use method of the above-described upper limb rehabilitation training system, as shown in FIG. 6. The method derives the coordinate data of the healthy arm by capturing the joints (including shoulder joints, elbow joints and wrist joints, etc.) of the user's healthy arm through the depth camera 11; then the coordinate data is filtered and processed to build a user motion model; the motion coordinates of the user's healthy arm are then converted to the motion coordinates of the affected arm through a mirror coordinate transformation, and the motion angle of the joints of the exoskeleton robotic arm are calculated through inverse kinematics solutions; through the execution of the servo control system of the arm, the exoskeleton robotic arm drives the user's affected arm to make symmetrical motion with the healthy arm.

Further, the motion postures of the user's healthy arm specifically comprise one or more of the following: the shoulder joint, the elbow joint, and the wrist joint of the healthy arm. The depth camera 11 can capture the coordinates of the user's neck, abdomen, and shoulder joint of the affected arm as needed.

Specific steps are shown in FIG. 6: Firstly, capturing the user's motion through the depth camera 11 to obtain the position information of each joint of the user (e.g., the coordinates of the shoulder joint, the elbow joint and the wrist joint of the healthy arm and the coordinates of the neck, the abdomen, and the shoulder joint of the affected arm), which is the coordinate data of the position where each joint is located in the spatial coordinate system. Since there is a large amount of such data and have certain jumps, it is necessary to filter these data to pick out reasonable and valid data. After filtering the coordinate data, a user motion model is created and a model of the human body is generated, and then mirrored coordinate data are formed through mirror coordinate transformation of the data, i.e., the motion coordinates of the healthy arm are converted to the motion coordinates of the affected arm by mirror coordinate transformation. Then, calculating the angle and position relationships between the joints of the exoskeleton robotic arm through the inverse kinematics solution and sends them to the robotic arm servo control system, which controls the robotic arm to perform the corresponding motion by means of the robot arm servo control system, so that the exoskeleton robotic arm drives the affected arm to make a symmetrical motion with the healthy arm. Preferably, the rehabilitation training system in this embodiment further comprises an absolute encoder. The absolute encoder is connected to the robotic arm servo control system for closed-loop feedback to determine whether each joint of the robotic arm is effectively moved to the exact position, thus forming a closed-loop control.

The use method of the upper limb rehabilitation training system described above enables symmetrical coordinated motion of the affected arm and the healthy arm. Based on similar principles and steps, non-mirror-symmetric coordinated motion can be achieved. The non-mirror-symmetric coordinated motion and mirror-symmetric coordinated motion have different algorithms in coordinate transformation processing, but the other processing steps and principles are the same. Specifically, when performing non-mirror-symmetric coordinated motion, the above-mentioned step “then mirrored coordinate data are formed through mirror coordinate transformation of the data, i.e., the motion coordinates of the healthy arm are converted to the motion coordinates of the affected arm by mirror coordinate transformation” should be adjusted to “the coordinates of the coordinated motion are then formed by coordinated motion coordinate transformation of the data, i.e., the motion coordinates of the healthy arm are converted to the motion coordinates of the affected arm that should be coordinated with the healthy arm through the coordinated motion coordinate transformation”. As a result, coordinated motion of the affected arm and the healthy arm can be realized, e.g. non-mirror coordination motion such as grasping with both hands and steering wheel control etc.

The user can drive his affected arm through the healthy arm for rehabilitation training. The postures of the healthy arm are collected by the depth camera 11, the exoskeleton robotic arm servo control system controls the exoskeleton robotic arm in real time to drive the affected arm to perform mirror symmetric rehabilitation motion and two-handed coordinated rehabilitation motion (for example, the user's left arm is the healthy arm and the right arm is the affected arm. When the user goes to the left side to fetch an object overhead, the healthy arm is lifted to the upper left, and the exoskeleton robotic arm controls the affected arm to lift to the upper left synchronously; controlling a steering wheel to simulate the action of steering wheel rotation; and some other coordinated motion). The invention performs real-time synchronized mirroring motion, capturing the user's healthy arm while calculating the motion trajectory of the affected arm in real time, and the motion of the affected arm is driven by the exoskeleton robotic arm, which is good in real time. It enables the user to do rehabilitation training actively, using his healthy arm to move autonomously, and the system controls the exoskeleton robotic arm to drive the affected arm to make symmetrical motion, and this method can help the user to recover better.

The motion control unit has a speed control function. When the current motion speed of the exoskeleton robotic arm is greater than a pre-set value, the motion control unit controls the exoskeleton robotic arm to reduce the motion speed. Since the rehabilitation process cannot be too fast, for safety, if the healthy arm is too fast during the mirror rehabilitation process, the affected arm should adopt a control strategy of speed limitation to reduce the speed and smooth the process.

A plurality of motion scenarios (e.g., picking an apple, kicking a ball, eating etc.) and/or interactive scenarios are stored in the central processor, specifically, a plurality of motion scenarios are stored in the storage module of the central processor, and the display 10 is used to display the plurality of motion scenarios and/or interactive scenarios. Among them, the multiple motion scenarios are used to be imitated or viewed by the user, and the interactive scenarios are used to interact with the user. This embodiment contributes to upper limb rehabilitation training by adding multiple factors such as visual and brain neurostimulation. Preferably, the motion scenarios comprise interactive simulation scenarios. The present embodiment provides multiple interactive simulation scenarios to increase interactive visual stimulation to solve dull motion during the rehabilitation training and has good rehabilitation training effect.

In one embodiment of the present invention, by combining the above-mentioned central processor having the visual stimulation function with the mirror rehabilitation training mode, the user can drive the affected arm with his own healthy arm, and in the process of rehabilitation, adding subjective consciousness factors of the user in combination with certain visual stimulation, it stimulates the user's active motion consciousness with the help of visual thus better rehabilitation training can be achieved. The upper limb rehabilitation training system in this embodiment is a body function training system, the visual stimulation is added in the process of rehabilitation to perform a certain degree of behavioral and psychological intervention. In addition, visual stimulation training such as fun and cognitive thinking can be added to the rehabilitation process to avoid dullness and inefficiency of traditional interventions. The upper limb rehabilitation robot in the present invention is provided with a plurality of built-in rehabilitation programs in combination with corresponding visual rehabilitation scenarios, which can provide better visual stimulation during the rehabilitation training process.

There are many different methods for using the upper limb rehabilitation robot in the present invention, according to whether the exoskeleton robotic arm provides assistances and the amount of assistance, three methods are provided as follows:

The first method is that the exoskeleton robotic arm provides all assistance, and the exoskeleton robotic arm drives the arm to move, which can realize the user's passive rehabilitation training mode; the second method is that the exoskeleton robotic arm partially assists the user, and the user's arm and the exoskeleton robotic arm apply force together, which can realize the passive assisted rehabilitation training mode; the third method is that the robotic arm does not apply assistance to the user, but receives the user's force and moves accordingly under the drag of the user, which can realize the active rehabilitation training mode.

The first method, i.e. passive rehabilitation training mode, is mainly for users with serious upper limb disability in the early stage to better restore muscle strength training and realize rehabilitation motion of single-joint and multi-joint in combination with visual application scenarios such as eating, grasping objects, and wiping tables etc.

Specifically, in the passive rehabilitation training mode, the three drive units 7 drive the arm puller 15 and the forearm puller 16 to move thus to further drive the arm to perform corresponding movements. It includes single joint rehabilitation training and multi-joint linked rehabilitation training. The display 10 displays rich motion scenarios, such as eating, grasping, glass cleaning etc., to visually stimulate the user when the user performs the movements.

More specifically, the passive rehabilitation training mode in this embodiment uses a visual synchronization method to provide targeted rehabilitation training to the user through 3D tutorials from the physician (setting the motion range of rehabilitation of the exoskeleton robotic arm by dragging the arm of the 3D figure model with a mouse) or parameterization (manually entering the rehabilitation motion angle of each joint). In the process of execution, the 3D figure model in the same motion posture as the exoskeleton robotic arm is played simultaneously, or an active view is set to play an arm motion scenario simulation of the same motion posture as the exoskeleton robotic arm (for example, matching scenarios such as eating, wiping the table, fetching overhead and other scenarios according to the motion), or game training. Specifically, in the passive rehabilitation training mode, the integrated joints are controlled using the control mode of a position ring, which controls each joint to perform absolute angles. As shown in FIG. 7, the central processor receives instructions of the joints' positions from the doctor, sends the angle information of the joints' motion to the robotic arm servo control system, the robotic arm servo control system calculates the time to do the motion and plans the route, and feedbacks the time parameters to the central processor, which performs synchronized motion matching of the 3D model, thus achieving the synchronization effect of the exoskeleton robotic arm and the video model's motion. The absolute encoder is connected to the robotic arm servo control system for closed-loop feedback to judge whether each joint of the robotic arm is effectively moved to the exact position, forming a closed-loop control.

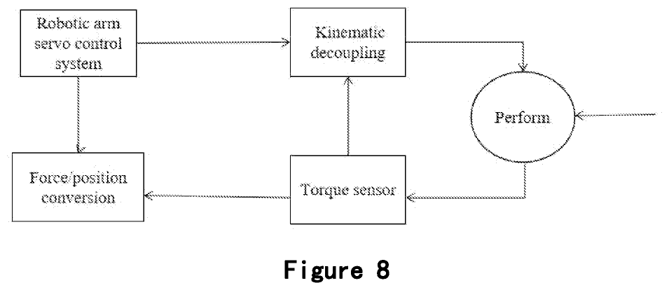

The second method, i.e. the passive assisted training mode, as shown in FIG. 8, is suitable for certain rehabilitation training, muscle strength recovery to a certain degree or mildly disabled users. The user's arm drives the movement of the exoskeleton robotic arm, the exoskeleton robotic arm determines the intention of the arm's movement, and cooperating with an assistance, so as to give a certain amount of compensatory movement. A certain visual stimulation can also be given to achieve better rehabilitation results. The drive unit 7 is controlled in a torque mode, and the user's arm drives the movement of the exoskeleton robotic arm, which determines the intention of the arm's movement and gives assistance. The integrated drive joint in this invention has a built-in one-dimensional torque sensor, and the movement intention of the three main joints is determined by separate sensor signals.

Among this, the determination of the intention is through a force sensor determining that the current force has changed. If there is a upward force it is determined that the arm intends to lift up, the exoskeleton robotic arm will assist to lift up the arm to a corresponding angle. If there is a downward pressure it is determined that the arm intends to put down, the exoskeleton robotic arm will assist to sink to a corresponding angle. There are two ways to determine the intention of the arm, one is to measure the change of the current torque value of each joint through the torque sensor in the integrated joint to determine the movement intention of the arm; the other is to compare and calculate the change of the difference between the absolute encoder and the incremental encoder in the integrated joint and then convert to the torque value. The motion direction and distance of the joint are calculated by judging the increase or decrease of the torque. The signals collected by the torque sensor are used for the hybrid control of force/position of the exoskeleton robotic arm. This torque information is kinematically decoupled and transformed into a motion deviation signal given by each degree of freedom to control the motion of the exoskeleton robotic arm, thus fusing the user's motion torque on the exoskeleton robotic arm into the closed-loop control of the exoskeleton robotic arm.

That is, the exoskeleton robotic arm using passive assisted mode, the joints of the robotic arm are equipped with detection sensors which detects the user force and direction in real time, when the user force exceeds a preset value, controls the exoskeleton robotic arm to assist the user in the direction of the force.

The third method, i.e. the active rehabilitation training mode, allows users to drag the robotic arm by themselves to do the strength recovery training by setting a resistance mode of each joint of the robotic arm. The active rehabilitation training mode is for users with better rehabilitation effect. The users can do fast movements with large amplitudes by setting a series of games with different difficulties and the users complete the game movements by swinging the arm. The active rehabilitation training mode can be combined with the exoskeleton robotic arm, it can also be used without the exoskeleton robotic arm, using only the depth camera 11 (i.e., the vision system performs the training independently). By setting a plurality of animated scenes, the user can perform interactive rehabilitation training games, and the user's movements are captured by the depth camera 11 to determine the movements.

More specifically, the active rehabilitation training mode may be realized in two ways, one is the arm dragging the robotic arm to move thus to achieve rehabilitation training in different levels and with different strengths by setting different resistance modes for each joint. For example, it can be a resistance mode using current loop control mode, different resistance modes can also be set, and the user can easily or forcefully drag the exoskeleton robotic arm, and some game training can be performed in this way. In this embodiment, the upper limb rehabilitation training system in this invention is a rehabilitation device that can adjust the damping strength, a mechanical rehabilitation physiotherapy equipment. Rehabilitation training in different levels and with different strengths can be achieved by setting different resistance modes for each joint, thus helping users restore the strength of the limb.

The other one is that the user can carry out the game rehabilitation training through arm motion postures captured by depth vision without the exoskeleton robotic arm. By setting game scenarios, the spatial coordinates of the game are matched with the coordinates of the end position of the arm, which can realize 2D/3D game interaction.

Among them, the motion control unit is connected to the central processor for controlling the motion of the exoskeleton robotic arm. Specifically, when in the passive rehabilitation training mode, the drive units drive the arm puller 15, forearm puller 16 to move, so as to drive the user's arm to carry out corresponding motion; when in passive assisted training mode, the drive unit is in the torque control mode, the user's arm drives the exoskeleton robotic arm to move, and the exoskeleton robotic arm determines the intention of the arm's movement, and assists with a force.

In summary, passive, passive assisted, active, mirror and collaborative rehabilitation training modes can be achieved in the upper limb rehabilitation training system in the present invention, and the system can be applied widely. The upper limb rehabilitation training provided in the present invention can also be used in community rehabilitation physiotherapy centers and community rehabilitation training centers.

In this embodiment, the upper limb rehabilitation training system comprises a functional electrical stimulator, the functional electrical stimulator comprises an electrical stimulation pulse generator, electrode pads and a microcomputer MCU, the microcomputer MCU is connected to said electrical stimulation pulse generator, a plurality of electrode pads are connected to the electrical stimulation pulse generator, and the electrode pads are used to fit at the upper limb of the user. This embodiment uses functional electrical stimulation to electrically stimulate the user's muscles using triangular or square wave micro-currents to enhance the strength and endurance of the muscles which improves the user's mobility and helps to improve the effect of rehabilitation.

The functional electrical stimulation stimulates the motor nerves of the user's muscles through epidermal and implantable electrodes. The electrical field between the electrodes generates a trigger potential on the nerve, which is chemically transmitted to the muscle cells via neuronal contacts and causes muscle contraction, resulting in muscle actions, which can be controlled by varying the voltage and frequency of the stimulation.

The invention also has a rehabilitation training and assessment system that intervenes in behavior, psychology and cognition.

The above embodiments are preferable embodiments of the present invention, but the implementation of the present invention is not limited by the above embodiments. Although detailed description has been given based on the embodiments, the skilled person should understand that: any other changes, modifications, alternatives, combinations, simplifications made without deviating from the spirit and principle of the present invention shall be equivalent substitutions and are included in the scope of protection of the present invention.

What is claimed is:

1-14. (canceled)

15. An upper limb rehabilitation training system comprising:
an upper limb function assessment device, wherein the upper limb function assessment device comprises a display, a depth camera and a central processor, wherein the depth camera is configured to capture a user's motion, the display is configured to display motion demonstration and the user's motion and the central processor is, respectively, connected to the display and the depth camera, the depth camera comprises an RGB camera and a depth camera, wherein the RGB camera is configured to obtain two-dimensional coordinates of the user's joints and the depth camera is configured to obtain depth coordinates of the user's joints, which comprises: first, a deep learning method based on a deep neural network is adopted to acquire the two-dimensional coordinates of the user's joints from color images captured by the RGB camera of the depth camera, and then the depth coordinates of the user's joints are acquired by the depth images captured by the depth camera of the depth camera, and finally the acquired two-dimensional coordinates and depth coordinates of the user's joints are mapped to the three-dimensional coordinates of the user's joints, in the deep learning method based on the deep neural network, self-obscuring images and images of hard-to-detect motion such as spin-forward and spin-backward are added as training sets to train the deep neural network model, wherein the self-obscuring means that one of the user's joints captured by the depth camera is obscured by other joints of the user itself, so as to avoid the problem of inaccurate detection of joints' coordinates due to self-obscuring, and accurately detect the 3D coordinates of the user's joints;
an exoskeleton robotic arm and a motion control unit, wherein the motion control unit is connected to the central processor for controlling the motion of the exoskeleton robotic arm, wherein the exoskeleton robotic arm further comprises an arm puller and a forearm puller, and the length of the arm puller and the forearm puller can be adjusted manually or electrically to accommodate users with different arm lengths, and the shoulder joint and elbow joint of the user correspond to the position of the shoulder joint and elbow joint of the exoskeleton robotic arm, respectively, wherein the user's motion comprises motion postures of the user's healthy arm and the depth camera is configured to capture the motion postures of the user's healthy arm in real time,
the central processor controls the motion of the exoskeleton robotic arm according to the motion postures of the user's healthy arm, thereby driving the user's affected arm on the exoskeleton robotic arm to make corresponding motion, the motion control unit controls three drive units for achieving abduction/adduction of the arm, lifting/lowering of the arm and flexion of the forearm of the exoskeleton robotic arm, respectively, the three drive units comprises a first drive unit corresponding to the first joint of the shoulder joint, a second drive unit corresponding to the second joint of the shoulder joint, and a third drive unit corresponding to the elbow joint; the first drive unit, the second drive unit and the third drive unit are configured to realize the abduction/induction of the arm, the lifting/lowering of the arm, and the flexion of the forearm, respectively, the exoskeleton robotic arm comprises a shoulder joint and an elbow joint, the shoulder joint comprises a first joint of the shoulder joint and a second joint of the shoulder joint and a third passive joint of the shoulder joint, and the elbow joint comprises a first joint of the elbow joint and a second passive joint of the elbow joint, the first joint of the elbow joint is configured to realize the flexion of the forearm, the third degree of freedom of the shoulder joint of the exoskeleton robotic arm is configured to achieve flexion of the forearm which is passively controlled;
the shoulder joint and the elbow joint of the exoskeleton robotic arm are of a surrounding sliding rail structure;
further, the central processor combines the assessment scales commonly used in clinical practice to perform the assessment by quantifying all the motion in the scales for automatic assessment which comprises a deep learning method based on Long Short-Term Memory to determine the completion of the motion, including: whether it is fully completed, partially completed or completely incomplete, during the training of the model, the key frames and key nodes of different motion sequences are manually marked to achieve static matching of standard motion, followed by automatic sampling to get more adjacent frames to achieve dynamic matching, and the key frames, key nodes and adjacent frames are combined for encoding to form a model of standard motion template sequences, finally, the longest common subsequence algorithm is used to identify whether the motion performed by the user conforms to the standard motion: firstly, the current motion of the user is detected in real time to form the motion sequence, and then the longest common subsequence is obtained by comparing the motion sequence with the standard motion template sequence model, so as to provide feedback on the non-standard degree of the current motion of the user, and to make judgment and specific scoring for the completion of the motion, the key frames, the key nodes, the adjacent frames and the current motion frames include the 3D coordinates of each joint of the user, after the assessment of all motion is completed, an assessment report is formed, and then the system and the physician provide a diagnosis plan and a targeted exercise prescription.

16. The upper limb rehabilitation training system according to claim 15, wherein a plurality of motion scenarios and/or interactive scenarios are stored in the central processor, and the display is configured to display the plurality of motion scenarios and/or interactive scenarios, wherein the plurality of motion scenarios are used for imitation or viewing by the user, and the interactive scenarios are used for interaction with the user.
